SEQUENCE LISTING (2 sequences)

SEQ ID NO: 1 - protein, 405 aa, Mus musculus
MRATDRMGARAVSELRLALLFVLVLGTPRLGVQGEDGLDFPEYDGVDRVINVNAKNYKNVFKKYEVLALLYHEPPEDDKASQRQFEMEELILELAAQVLEDKGVGFGLVDSEKDAAVAKKLGLTEEDSVYVFKGDEVIEYDGEFSADTLVEFLLDVLEDPVELIEGERELQAFENIEDEIKLIGYFKSKDSEHYKAYEDAAEEFHPYIPFFATFDSKVAKKLTLKLNEIDFYEAFMEEPMTIPDKPNSEEEIVSFVEEHRRSTLRKLKPESMYETWEDDLDGIHIVAFAEEADPDGYEFLETLKAVAQDNTENPDLSIIWIDPDDFPLLVPYWEKTFDIDLSAPQIGVVNVTDADSIWMEMDNEEDLPSADELEDWLEDVLEGEINTEDDDDDDDDDDDDDDDDD

SEQ ID NO: 2 - protein, 396 aa, Homo sapiens
MSATDRMGPRAVPGLRLALLLLLVLGTPKSGVQGQEGLDFPEYDGVDRVINVNAKNYKNVFKKYEVLALLYHEPPEDDKASQRQFEMEELILELAAQVLEDKGVGFGLVDSEKDAAVAKKLGLTEVDSMYVFKGDEVIEYDGEFSADTIVEFLLDVLEDPVELIEGERELQAFENIEDEIKLIGYFKSKDSEHYKAFEDAAEEFHPYIPFFATFDSKVAKKLTLKLNEIDFYEAFMEEPVTIPDKPNSEEEIVNFVEEHRRSTLRKLKPESMYETWEDDMDGIHIVAFAEEADPDGFEFLETLKAVAQDNTENPDLSIIWIDPDDFPLLVPYWEKTFDIDLSAPQIGVVNVTDADSVWMEMDDEEDLPSAEELEDWLEDVLEGEINTEDDDDDDDD

(19) United States
(12) Patent Application Publication
(10) Pub. No.: US 2026/0258103 A1
(43) Pub. Date: Sep. 3, 2026

(54) LUMINESCENT METAL BINDING PROTEINS AND PEPTIDES FOR USE IN MICROSCOPY

(71) Applicants: Photon Biosciences, LLC, Spokane Valley, WA (US); Montana State University, Bozeman, MT (US)

(72) Inventors: Kevin Lewis, Spokane, WA (US); ChulHee Kang, Pullman, WA (US); James A. Brozik, Pullman, WA (US); Charles Martin Lawrence, Bozeman, MT (US)

(21) Appl. No.: 19/163,834

(22) PCT Filed: Mar. 8, 2024
(86) PCT No.: PCT/US2024/019269

§ 371 Date: Sep. 9, 2025

Related U.S. Application Data
(60) Provisional application No. 63/489,725, filed on Mar. 10, 2023.

(51) Int. Cl. C07K 14/47; C12N 15/63; G01N 21/64; G01N 33/53

(52) U.S. Cl. CPC C07K 14/4728; C12N 15/63; G01N 21/64; G01N 33/5308; G01N 2333/4727

(57) ABSTRACT

Microscopy probes that include luminescent metal binding proteins and peptides and analogs thereof that are configured to be used in electron microscopy and methods of performing light and electron microscopy using the microscopy probes of the present technology.

### CROSS-REFERENCE TO RELATED APPLICATION

This application claims the benefit of U.S. Provisional Patent Application No. 63/489,725, filed on Mar. 10, 2023, which is incorporated by reference herein in its entirety.

### STATEMENT REGARDING FEDERALLY SPONSORED RESEARCH OR DEVELOPMENT

The present technology was made with government support under 1R41GM148117-01 awarded by the Department of Health and Human Services. The government has certain rights in the invention.

### SEQUENCE LISTING

This application contains a ST.26 compliant Sequence Listing, which was submitted in XML format via Patent Center, and is hereby incorporated by reference in its entirety. The XML copy, created on Mar. 7, 2024, is named “2024-03-07 1490648001US00 Sequence Listing” and is 4 KB in size.

### TECHNICAL FIELD

The present technology generally relates to luminescent metal binding proteins and peptides and methods of using the luminescent metal binding proteins and peptides for light microscopy, electron microscopy, and correlative light and electron microscopy.

### BACKGROUND

Microscopes are important tools for understanding small-scale phenomena, especially cellular functions. There are two fundamentally different types of microscopes: light microscopes, which use glass lenses to focus light on a specimen to form an image, and electron microscopes, which use electromagnetic lenses to focus beams of electrons on a specimen. Each microscope is more suited to particular applications. Living and non-living specimens can be viewed with a light microscope, where only non-living specimens can be viewed with an electron microscope, so light microscopes can be more helpful for understanding cell function in living tissues. On the other hand, electron microscopes allow nearly 1000× greater resolution than light microscopes, meaning that electron microscopes are preferred for detecting smaller structures like viruses, organelles, and globular proteins and peptides.

Correlative light and electron microscopy (CLEM) is a two-part technique that allows the analysis of the same specimen with a combination of light and electron microscopy tools. Many CLEM techniques use a luminescent (i.e., light-emitting probe) to identify a molecular or cellular structure under a light microscope, and then an electron microscope is used to identify the same structure under higher resolution. Some CLEM techniques use a probe that has both luminescent (i.e., light-emitting) and electron dense properties.

Some CLEM techniques use a fusion of two proteins or peptides, one of which is fluorescent and the other is electron dense. The combination of these two proteins or peptides with different folding kinetics, protein biochemistry, and other characteristics can lead to complications under different intracellular environments. Furthermore, because fluorescent proteins are derived from a handful of precursor proteins and because there are a few proteins or peptides that can be used as electron dense probes, viable CLEM probes are limited. Additionally, many of the probes contain or use expensive and/or toxic metals, such as cadmium and gold, as contrasting agents.

Therefore, there is need in the art for improved microscopy probes for light microscopy, electron microscopy, and CLEM.

### BRIEF DESCRIPTION OF THE DRAWINGS

Many aspects of the present technology can be better understood with reference to the following drawings. The features in the drawings are not necessarily drawn to scale. Instead, emphasis is placed on clearly illustrating the principles of the present technology. Furthermore, certain features can be shown as transparent/partially transparent in certain views for clarity of illustration only and not to indicate that the component is necessarily transparent. Features may also be shown schematically.

FIGS. 1A-1E show the structural properties of mouse calsequestrin (MmCasq1). In particular, FIG. 1A shows the protein sequence of MmCasq1. FIG. 1B shows the three domains (Domain I, II, and III) of MmCasq1. FIG. 1C shows an example MmCasq1 oligomer.

FIG. 1D shows the calcium-binding profile of MmCasq1. FIG. 1E shows the size-exclusion elution profile of MmCasq1 at different calcium concentrations and the corresponding oligomeric state of the protein, which was determined by multiangle static light scattering.

FIGS. 2A-2F show MmCasq1 binding sites and kinetics. In particular, FIG. 2A shows the normalized mainchain atomic displacement obtained from X-ray crystallographic data, and illustrates three to four structural Ca²⁺-binding sites of MmCasq1, named high-affinity site A (four black diamonds), high-affinity site B (three dark gray squares), high-affinity site C (FIG. 2A, three dark gray diamonds), and high-affinity site D (FIG. 2A, three dark gray triangles), inner sphere ligands for Ca²⁺ and outer sphere ligands for Nd3+(black circles), inner sphere ligands for both Ca²⁺ and Nd³⁺ (light gray circle), outer sphere for Ca²⁺ but inner sphere for Nd³⁺ (dark gray circle).

FIG. 2B shows two Ca²⁺ binding sites (large spheres are Ca²⁺) of MmCasq1.

FIG. 2C shows how the binuclear Ca²⁺ binding sites of FIG. 2B change to mononuclear binding sites for Nd³⁺ (large sphere represents Nd³⁺).

FIG. 2D shows MmCasq1's Ln³⁺ binding affinity by luminescence titration. Sites were classified by affinity as either strong (129 pM) or weak (1.16 μM). FIG. 2E shows Tb³⁺-Casq1 excitation and emission spectra. FIG. 2F shows Tb³⁺-Casq1 luminescence lifetime. The lifetime constant (i) was determined to be 1501 s.

FIGS. 3A and 3B show luminescence of Human calsequestrin (HsCasq1) and MsCasq1 with a Gly/Ser linker and a strep-tag expressed in E. coli and P. aeruginosa. In particular, FIG. 3A shows Eu³⁺ luminescence (right) over non-specific signal (left) in E. coli expressing HsCasq1. FIG. 3B shows Eu³⁺ luminescence (right) over non-specific signal (left) in P. aeruginosa PAO1 expressing MmCasq1.

FIGS. 4A-4D show visualization of MmCasq1 by cryo-ET in four successive sections along Z (each 4.6 nm thick) through a representative sub tomographic volume (50×50×20 nm). A cluster of five MmCasq1 probes is clearly visible as black punctate points 2-3 nm in diameter, extending left to right across the second and third sections of the tomogram (FIGS. 4B and 4C). The position of the left most particle is denoted by the red arrow in FIG. 4B. Acquisition parameters were as follows: magnification 36,000×; dose symmetric tilt range+/−60 degrees; tilt increment 3 degrees; total dose 60 e-per square Angstrom; pixel size 0.115 nm/px; defocus −3.0 μm. Tomograms were reconstructed using IMOD 4.11.

FIG. 5 is a block diagram illustrating an example CLEM method in accordance with an embodiment of the present technology.

### DETAILED DESCRIPTION

The present technology is generally directed to microscopy probes that comprise luminescent metal binding proteins, peptides, and/or analogs thereof that are configured to be used in electron microscopy. The present technology is also directed to methods of performing microscopy using the microscopy probes of the present technology.

As used herein, “microscopy probe” and “microscopy probes” includes any molecule that can be used to distinguish a feature, structure, or event during microscope imaging. Microscopy probes include probes that can distinguish a feature, structure, or event using any kind of microscope, including but not limited to simple microscopes, compound microscopes, scanning electron microscopes, transmission electron microscopes, stereomicroscopes, or scanning probe microscopes. A single microscopy probe can be used to distinguish a feature, structure, or event using more than one type of microscope. A microscopy probe can also distinguish one particular feature, structure, or event; or multiple features, structures, or events. For example, a microscopy probe can be used to label a particular type of protein in a cell (for example actin proteins) or it can be used to label many structures (for example acidic bodies). A microscopy probe can be a luminescent probe and/or an electron dense probe.

As used herein, a “luminescent” probe is a probe that emits light by a means other than heat; and it includes both fluorescent probes and phosphorescent probes. As used herein, a “fluorescent” probe is a probe that absorbs electromagnetic radiation and emits electromagnetic radiation at a different wavelength. A “phosphorescent” probe absorbs electromagnetic radiation and, after some time, emits the electromagnetic radiation at a different wavelength. A phosphorescent probe can be detected using a fluorescent microscope.

As used herein, “light microscopy” includes microscopy that uses light waves to detect a microscopy probe. “luminescent microscopy,” “photoluminescent microscopy,” “photoluminescence microscopy” or “fluorescent microscopy” use light waves to detect a luminescent microscopy probe.

As used herein, an “electron dense” probe is a microscopy probe that has one or more electron dense structures and is therefore configured to be used as a probe for electron microscopy. As used herein “electron microscopy” includes any microscopy technique that uses an electron beam to detect an electron dense probe. Electron microscopy includes scanning electron microscopy (SEM), and transmission electron microscopy (TEM), including cryoelectron tomography (CryoET) and scanning TEM.

As used herein, a “luminescent metal binding protein or peptide” is a protein or peptide that binds or chelates to a luminescent metal. Luminescent metal binding proteins and peptides include lanthanide or actinide binding proteins and peptides that bind to luminescent lanthanides or actinides including, for example Ce³⁺, Pr³⁺, Nd³⁺, Pm³⁺, Sm³⁺, Eu³⁺, Gd³⁺, Tb³⁺, Dy³⁺, Ho³⁺⁺, Er³⁺, Tm³⁺, Yb³⁺, Lu³⁺, U³⁺, U⁴⁺, U⁵⁺, or U⁶⁺. Luminescent metal binding proteins and peptides also include actinide binding proteins that bind to luminescent actinides.

The elements described in this technology are abbreviated according to their symbols on the periodic table. For example, Ca (Calcium), Eu (Europium), Pr (Praseodymium), Nd (Neodymium), Tb (Terbium), Ce (Cerium), Pm (Promethium), Sm (Samarium), Gd (Gadolinium), Dy (Dysprosium), Ho (Holmium), Er (Erbium), Tm (Thulium), Yb (Ytterbium), Lu (Lutetium), and U (Uranium), among others, are described in this technology. Elements may be described followed immediately by their charge. For example, Eu³⁺ is a europium ion with a charge of 3+.

As used herein, an “analog” of a molecule is a variation of the molecule where the structure has been modified. Structural modifications include but are not limited to additions, deletions, or substitutions of atoms or molecules, including functional groups, amino acids, peptides, and proteins.

### Microscopy Probes Comprising Luminescent Metal Binding Proteins or Peptides

The present technology includes microscopy probes comprising a luminescent metal binding protein or peptide that are configured to be used for both light microscopy and electron microscopy, and thus for CLEM.

The luminescent metal binding protein or peptide may be genetically engineered. As used herein, “genetically engineered” proteins and peptides are non-naturally occurring proteins and peptides. In some embodiments, the present technology includes methods of using both non-naturally occurring (i.e., genetically engineered) and naturally occurring luminescent metal binding proteins or peptides.

In some embodiments, for example, the luminescent metal binding protein or peptide is an electron dense probe. A luminescent metal binding protein or peptide may bind to a luminescent metal. For example, the luminescent metal binding protein or peptide may bind to an electron dense luminescent metal. The luminescent metal binding protein or peptide may bind to a luminescent metal having an atomic number of at least 57. The luminescent metal binding protein or peptide may bind to a luminescent lanthanide. The luminescent metal binding protein or peptide may bind to a luminescent actinide. In some aspects, the luminescent metal binding protein is a lanthanide binding protein or lanthanide binding peptide. For example, the luminescent metal binding protein or peptide may be configured to bind to Ce³⁺, Pr³⁺, Nd³⁺, Pm³⁺, Sm³⁺, Eu³⁺, Gd³⁺, Tb³⁺, Dy³⁺, Ho³⁺⁺, Er³⁺, Tm³⁺, Yb³⁺, Lu³⁺, U³⁺, U⁴⁺, U⁵⁺, or U⁶⁺. In some aspects, the luminescent metal binding protein is a lanthanide binding protein or analog thereof. In some aspects, the luminescent metal binding protein or peptide is a lanthanide binding peptide (LBT) or analog thereof.

In some embodiments, the luminescent metal binding protein or peptide has a highly negatively-charged surface. The negatively-charged surface may facilitate the binding or chelation to a luminescent metal. As such, the luminescent metal binding protein or peptide may comprise a plurality of negatively charged amino acids. In particular, the luminescent metal binding protein or peptide may comprise a high content of the amino acids Aspartate (D) and Glutamate (E). For example, the luminescent metal binding protein or peptide may comprise an amino acid sequence having more than 20% D and E amino acids. The luminescent metal binding protein or peptide may comprise an amino acid sequence having more than 25% D and E amino acids. The luminescent metal binding protein or peptide may comprise an amino acid sequence having more than 30% D and E amino acids.

In some embodiments, the luminescent metal binding protein or peptide is calcium binding protein or analog thereof. The calcium binding protein or peptide or analog thereof may be configured to bind to one or more luminescent metals. For example, the luminescent metal binding protein or peptide may be a calbindin, calmodulin, calsequestrin, troponin, calretinin, or analog thereof.

In some embodiments, the luminescent metal binding protein or peptide is a homolog, ortholog, paralog, or analog of calsequestrin. In some aspects, the luminescent metal binding protein or peptide may be a human calsequestrin protein or an ortholog thereof (a calsequestrin from another species; i.e. chimpanzee, cow, rat, mouse, etc.). The luminescent metal binding protein may also be a paralog of calsequestrin. For example, the luminescent metal binding protein may be calsequstrin 1 (Casq1) or calsequestrin 2 (Casq2). In some embodiments, the luminescent metal binding protein comprises a mouse calsequestrin (MmCasq1) or analog thereof. The luminescent metal binding protein may comprise a sequence that is at least 80%, 85%, 90%, 95%, or 98% identical to SEQ ID NO: 1. In some aspects, the luminescent metal binding protein or peptide may be a human calsequestrin (HsCasq1) or analog thereof. In some embodiments, the luminescent metal binding protein comprises a sequence that is at least 80%, 85%, 90%, 95%, or 98% identical to SEQ ID NO: 2.

In some embodiments, the present technology includes any of the metal binding proteins or peptides that are disclosed in International (PCT) Patent Pub. No. WO 2018/187303, which is incorporated by reference herein in its entirety.

In some embodiments, the luminescent metal binding protein or peptide may be an analog of a naturally occurring or synthetic luminescent metal binding protein. The luminescent metal binding protein or peptide may have one or more modifications that promote its function, for example the modifications may improve the luminescent metal binding, solubility, or localization, of the protein or peptide. The luminescent metal binding protein may have functional groups, amino acids, or peptide sequences that are added, deleted, substituted, or modified with respect to the luminescent metal binding protein or peptide from which it is derived. In some embodiments, the luminescent metal binding protein or peptide may comprise a tag to aid in purification or isolation of the luminescent metal binding protein or peptide. For example, the luminescent metal binding protein or peptide may comprise a His-tag, C-Tag, strep-tag, GST, or other tag known in the art.

In some embodiments, the microscopy probe comprises a luminescent metal binding protein or peptide that is fused to a secondary protein or peptide to form a fusion protein or fusion peptide. The secondary protein or peptide may promote the function of the microscopy probe. For example, the secondary protein or peptide may improve luminescent metal binding protein's solubility, localization, or ability to bind a luminescent metal. The luminescent metal binding protein may be fused to a secondary protein or peptide that promotes specific localization. For example, the luminescent metal binding protein or peptide may be fused to a nuclear localization signal, a protein binding domain, or a transmembrane protein. In some aspects, the secondary protein or peptide may provide an additional function in addition to the function provided by the luminescent metal binding protein. For example, the luminescent metal binding protein or peptide may be fused to an enzyme or a fluorescent protein. In some embodiments, the luminescent metal binding protein or peptide may be fused to a secondary protein or peptide with a linker between. For example, the luminescent metal binding protein or peptide may be fused to a secondary protein with a Gly/Ser linker between.

In some embodiments, the luminescent metal binding protein or peptide is configured to bind a plurality of luminescent metals. For example, the luminescent metal binding protein or peptide may be configured to bind between 2-100 luminescent metal ions. In some embodiments, the luminescent metal binding protein or peptide may bind 10-20, 20-30, 30-40, 40-50, 50-60, 60-70, 70-80, 80-90, or 90-100 metal ions. In some embodiments, the luminescent metal binding protein or peptide may bind more than 10 metal ions, more than 20 metal ions, more than 30 metal ions, more than 40 metal ions, or more than 50 metal ions. In some embodiments, the luminescent metal binding protein or peptide may be configured to form oligomers, where each monomer of the oligomer binds more than 2 metal ions, more than 4 metal ions, more than 6 metal ions, more than 8 metal ions, or more than 10 metal ions.

In some embodiments, the luminescent metal binding protein or peptide is configured to form an oligomer with other luminescent metal binding proteins or peptide. For example, the luminescent metal binding protein or peptide may form a dimer, trimer, or tetramer with other luminescent metal binding proteins. In some embodiments, the luminescent metal binding protein or peptide is configured to form a polymer with other luminescent metal binding proteins or peptides.

In some embodiments, the luminescent metal binding protein or peptide is a concatenated construct. As used herein, a “concatenated construct” includes proteins or peptides containing two or more luminescent metal binding proteins or peptides fused into a single polypeptide chain.

In some embodiments, the luminescent metal binding protein or peptide is configured to be phosphorescent. In some embodiments, the luminescent metal binding protein or peptide is configured to be fluorescent. The luminescent metal binding protein or peptide may be excited by UV, visible, or near-infrared light. The luminescent metal binding protein or peptide may emit from UV to infrared light. The luminescent metal binding protein or peptide may be configured to be excited by a wavelength of 250 to 1000 nm and emit as multiple bands within a useful range of 300 to 1600 nm.

Luminescent metal binding proteins or peptides may be configured to be excited by a wavelength of the luminescent metal that is bound to the luminescent metal binding protein or peptide, for example at the wavelengths (λₑₓ) listed in table 1. Luminescent metal binding proteins or peptides may subsequently emit light at a wavelength of the luminescent metal that is bound to the luminescent metal binding protein or peptide, for example at the wavelengths (λₑₘ) listed in Tal 1.

TABLE 1
Excitation/Emission of Luminescent Metals

| | λₑₓ | |
|---|---|---|
| Symbol | (nm ± 5 nm) | λₑₘ (nm ± 5 nm) |
| La³⁺ | N/A | N/A (closed shell) |
| Ce³⁺ | 252 | 285, 300, 330, 380 |
| Pr³⁺ | 294 | 490, 530, 610, 645, 726, 890, 1060 |
| Nd³⁺ | 353 | 880, 1060, 1330 |
| Pm³⁺ | N/A | N/A (radioactive) |
| Sm³⁺ | 401 | 564, 601, 643, 706 |
| Eu³⁺ | 395 | 580, 590, 615, 650, 690, 710 |
| Gd³⁺ | 274 | 311 |
| Tb³⁺ | 375 | 490, 545, 590, 620, 650 |
| Dy³⁺ | 385 | 480, 575, 675 |
| Ho³⁺ | 450 | 550, 650, 746, 903, 1115 |
| Er³⁺ | 486, 514.5 | 547, 667, 1550 |
| Tm³⁺ | 356, 463 | 365, 455; 482, 650 |
| Yb³⁺ | 980 | 980 |
| Lu³⁺ | N/A | N/A (closed shell) |
| U³⁺ | 300 to 600 | N/A |
| U⁴⁺ | 245 | 289, 291, 318, 320, 335, 338, 345, |
| | | 394, 409, and 525 |
| U⁵⁺ | 255 | 440, 510 |
| U⁶⁺ | 230, 266, 275, | 480, 500, 520, 545, 570, 588 |
| | 308, 313 | |

In some embodiments, the microscopy probe comprising a luminescent metal binding protein or peptide is genetically encodable. As used herein, a “genetically encodable” protein or peptide can be produced in vivo or in vitro by transcription and translation of a polynucleotide or recombinant gene. The present technology includes a polynucleotide sequence that encodes a microscopy probe comprising a luminescent metal binding protein. In some aspects, a polynucleotide encoding a microscopy probe can be incorporated into a vector. For example, a polynucleotide encoding a microscopy probe can be incorporated into a plasmid, viral-based vector, phagemid-based vector, cosmid-based vector, yeast artificial chromosome vector. The vector can be used for propagation of the polynucleotide encoding the microscopy probe and/or for delivery of the polynucleotide encoding the microscopy probe into a cell or multicellular organism.

In some embodiments, the microscopy probe comprising a luminescent metal binding protein is configured to be detected using photoluminescence microscopy. For example, the microscopy probe may be configured to be detected using a widefield fluorescence microscope, a confocal fluorescence microscope, and a super-resolution fluorescence microscope. In particular, the microscopy probe may be configured to be detected using a fluorescent confocal microscope or light sheet microscope. The microscopy probe may be configured to be detected using multiphoton microscopy. The microscopy probe may be configured to be detected using super-resolution microscopy. The microscopy probe may be configured to be detected by first, exciting the microscopy probe with electromagnetic radiation and second, detecting the electromagnetic radiation that is emitted by the microscopy probe. In particular, the microscopy probe may be configured to absorb electromagnetic radiation at a first wavelength and emit electromagnetic radiation at a second wavelength. The detection of the microscopy probe may be done using any light microscopy technique known to one of ordinary skill in the art.

In some embodiments, the microscopy probe comprising a luminescent metal binding protein or peptide is configured to be sufficiently electron dense to be detected using an electron microscope without additional contrasting agents. The electron microscope may be either a scanning electron microscope or a transmission electron microscope. The electron microscope may collect data including the collections of tomographic tilt series. The electron microscope may collect data under room temperature or cryogenic conditions.

In some embodiments, the microscopy probe comprising a metal binding protein or peptide is configured to be detected using an electron microscope and electron energy loss spectroscopy (EELS) or energy dispersive X-ray spectroscopy (EDS).

In some embodiments, the microscopy probe comprising a metal binding protein or peptide is configured to be detected using X-ray Fluorescence (XRF), X-ray Fluorescence Microscopy, or X-ray Fluorescence Spectroscopy. The X-ray source may include synchrotrons, for synchrotron XRF, synchrotron X-ray Fluorescent Microscopy and synchrotron X-ray fluorescence microscopy.

In some embodiments, the microscopy probe comprising a metal binding protein or peptide is configured to be detected using atom probe tomography.

In some embodiments, the microscopy probe comprising a metal binding protein or peptide is configured to be detected using Nano-SIMS (Nano-secondary ion mass spectroscopy).

In some embodiments, the microscopy probe comprising a luminescent metal binding protein is configured to be detected using a light microscope and an electron microscope. The microscopy probe may comprise a portion that is sufficiently dense to be detected using an electron microscope and a portion that is configured to absorb electromagnetic radiation at a first wavelength and emit electromagnetic radiation at a second wavelength. The portion that is sufficiently dense to be detected using an electron microscope may be the same portion that is configured to absorb electromagnetic radiation at a first wavelength and emit electromagnetic radiation at a second wavelength. The microscopy probe may be configured to be detected using a light microscope and an electron microscope without additional contrasting agents. In some aspects, the microscopy probe is configured to be detected in both the light microscope and electron microscope steps of a CLEM technique.

In some embodiments, the microscopy probe comprising a luminescent metal binding protein or peptide is configured to be detected using a light microscope and X-ray fluorescence techniques, such as an X-ray fluorescence microscope. The microscopy probe may be configured to be detected using a light microscope and X-ray fluoresecne without additional contrasting agents. In some aspects, the microscopy probe is configured to be detected in both the light microscope and X-ray fluorescent microscope steps of a Correlated Light and X-ray Fluorescent Microscopy (CLXM) technique.

### Methods of Performing Light Microscopy, Electron Microscopy, and CLEM Using the Microscopy Probe Comprising a Luminescent Metal Binding Protein or Peptide

The present technology further includes methods of performing light microscopy, electron microscopy, and/or CLEM to detect the microscopy probe comprising a luminescent metal binding protein or peptide.

In some embodiments, the method comprises detecting the microscopy probe comprising a luminescent metal binding protein or peptide in vivo. The method may comprise generating a polynucleotide that encodes a microscopy probe. The method may comprise incorporating the polynucleotide that encodes a microscopy probe into a vector. For example, a polynucleotide encoding a microscopy probe may be incorporated into a plasmid, viral-based vector, phagemid-based vector, cosmid-based vector, yeast artificial chromosome vector. The method may include using the vector for propagation of the polynucleotide encoding the microscopy probe. The polynucleotide encoding a microscopy probe may be incorporated into a vector using any technique known to a person of ordinary skill in the art.

In some embodiments, the method includes delivering a vector comprising a polynucleotide encoding the microscopy probe comprising a luminescent metal binding protein or peptide into a cell or multicellular organism. The vector may be delivered into a cell or multicellular organism using any technique known to one of ordinary skill in the art. In some aspects, the method includes delivering a polynucleotide encoding a microscopy probe into a prokaryotic cell, a eukaryotic cell, or a multicellular organism. The polynucleotide encoding a microscopy probe may be delivered into a mammal. The polynucleotide encoding a microscopy probe may be delivered by transduction, transfection, electroporation, or microinjection. In some instances, the method includes using gene editing tools such as CRISPR/Cas9 to deliver a polynucleotide encoding a microscopy probe into a cell or multicellular organism.

In some embodiments, the method includes inducing expression of a polynucleotide encoding a microscopy probe comprising a luminescent metal binding protein or peptide. A polynucleotide encoding a microscopy probe may be induced in a cell or multicellular organism using any technique known to a person of ordinary skill in the art. The polynucleotide encoding a microscopy probe may be constitutively expressed or expressed using an inducible system. In some instances, transgenic species that express the polynucleotide encoding a microscopy probe may be generated. In some embodiments, the transgenic species may constitutively express the polynucleotide encoding a microscopy probe. In some embodiments, the transgenic species may express the polynucleotide encoding a microscopy probe through an inducible system. For example, the transgenic species may express the polynucleotide encoding a microscopy probe through a Cre recombinase-LoxP inducible system.

In some embodiments, the method comprises preparing the specimen comprising a microscopy probe comprising a luminescent metal binding protein or peptide for microscopy. As used herein, “specimen” includes cells, a multicellular organism, or a portion of a multicellular organism that comprise a microscopy probe comprising a luminescent metal binding protein or peptide. The specimen may be prepared for microscopy using any technique known to a person of ordinary skill in the art. For example, the method may comprise isolating the specimen, fixing the specimen, and/or sectioning the specimen. The specimen may be mounted on a suitable imaging surface, such as a slide or imaging dish. In some aspects, the specimen is kept live for live cell imaging.

In some aspects, the method includes processing the specimen for the detection of probes other than the microscopy probe of the present technology. For example, the specimen may be processed to detect immunofluorescent probes, contrasting agents, or dyes known to a person of ordinary skill in the art. In some aspects, a clearing technique may be used to enhance resolution of the microscopy probe.

In some embodiments, the method includes imaging a specimen in an imaging medium. As used herein, “imaging” refers to acquiring an image or data from a specimen using a microscope. In some aspects, the components of the imaging medium are selected based on criteria known to a person of ordinary skill in the art.

In some embodiments, the method includes imaging the specimen in an imaging medium comprising a luminescent metal. In some aspects, the imaging medium comprises a luminescent lanthanide or a luminescent actinide. For example, the imaging medium may comprise Ce³⁺, Pr³⁺, Nd³⁺, Pm³⁺, Sm³⁺, Eu³⁺, Gd³⁺, Tb³⁺, Dy³⁺, Ho³⁺⁺, Er³⁺, Tm³⁺, Yb³⁺, Lu³⁺, U³⁺, U⁴⁺, U⁵⁺, U⁶⁺, or other lanthanides and actinides.

The method may also include imaging the specimen in an imaging medium comprising a compound that prevents precipitation of the luminescent metal. The imaging media may comprise a compound that prevents precipitation of a luminescent lanthanide. For example, the imaging medium may comprise disodium α-glycerophosphate. The imaging medium may comprise disodium α-glycerophosphate instead of sodium phosphate.

In some aspects, the method includes imaging the specimen in an imaging medium comprising additional contrasting agents. The imaging medium comprises additional dyes or luminescent probes known to a person of ordinary skill in the art. For example, the imaging media may comprise a nuclear dye such as a Hoechst stain.

In some embodiments, the method includes imaging the specimen using a light microscope. The specimen may be imaged using any microscope or parameters known to one of ordinary skill in the art.

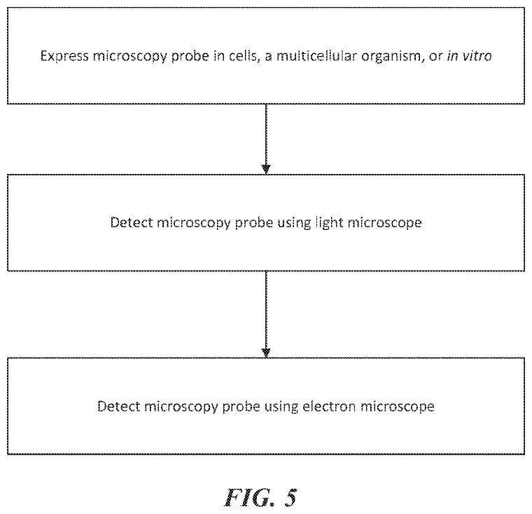

In some embodiments, the method comprises detecting the microscopy probe comprising a luminescent metal binding protein using an electron microscope after the microscopy probe has been detected on a light microscope. The method may comprise detecting the microscopy probe using a CLEM technique, for example, as shown in FIG. 5. The method may comprise detecting the microscopy probe using SEM or TEM, including CryoET techniques.

In some embodiments, the method comprises freezing a specimen having a microscopy probe that had been previously detected using a light microscope. The specimen may be frozen using any technique known to one of skill in the art. For example, the specimen comprising a microscopy probe may be flash frozen. The specimen comprising a microscopy probe may be frozen using liquid nitrogen, liquid ethane, liquid propane, liquid ethane propane mixtures or other flash freezing tools known to a person of ordinary skill in the art. The specimen comprising a microscopy probe may be flash frozen onto an electron microscope grid.

In some embodiments, the method comprises thinning or sectioning the specimen. The specimen comprising the microscopy probe is thinned or sectioned according to any technique known to one of ordinary skill in the art. For example, the specimen comprising a luminescent metal binding protein or peptide may be thinned or sectioned using a milling technique, such as focused ion beam (FIB) milling. The FIB mill may be used to make thinned or cross sections (lamelli) through the specimen, or lift outs from the specimen, comprising the luminescent metal binding protein. In some instances, the luminescence of the luminescent metal binding protein of the microscopy probe is used to determine which portion of the specimen to thin, section or otherwise mill. In some embodiments, the luminescent metal binding protein or peptide may be detected before sectioning, thinning, or milling. The luminescent metal binding protein or peptide may be detected during sectioning, thinning, or milling. The luminescent metal binding protein or peptide may be detected after sectioning, thinning, or milling. In some embodiments, the luminescent metal binding protein may be detected using block face imaging. In some embodiments, the luminescent metal binding protein or peptide may be imaged using electron microscopy without prior FIB milling.

In some embodiments, the method comprises using a light microscope to determine which region of interest on a cross sectioned specimen should be imaged using electron microscopy. A fluorescent or confocal microscope may be used to determine the region of interest. In some instances, a superresolution microscope can be used to determine the region of interest. The luminescent metal binding protein may be detected to identify the region of interest. 2D or 3D images may be taken with a light microscope (e.g., fluorescent, confocal, or superresolution microscopes) and compared to subsequent 2D and 3D electron microscope images. Comparing light microscope images and electron microscope images enable a user to correlate features of the light microscope images to features of the electron microscope images. In some embodiments, 2D or 3D luminescent templates are used to search the EM subvolumes. In such embodiments, once a luminescent metal binding protein or peptide is localized within the subvolumes, the immediate vicinity may be searched for the molecule of interest. In some embodiments, template matching algorithms or similar strategies are used to yield information on the orientation, assembly state, and/or conformation of the target protein or peptide within the micrograph or tomogram.

In some embodiments, the method includes imaging a specimen using an electron microscope. The specimen may be imaged using an electron microscope after the specimen had been imaged using a light microscope. In some instances, the specimen may be imaged using an electron microscope without previously imaging using a light microscope. In some embodiments, the specimen may be imaged using an electron microscope with an embedded light microscope. In some aspects, the specimen comprising a luminescent metal binding protein is imaged using a scanning or transmission electron microscope.

In some embodiments, micrographs and/or tomograms are searched for electron dense particles to localize individual fusion protein molecules. The search may be limited to subvolumes initially identified by light microscopy or super-resolution light microscopy at the single molecule level. In some embodiments, the 2D images or 3D tomograms collected using the electron microscope are matched with 2D or 3D images that were taken of the same sample with a fluorescent, confocal, or superresolution microscope. The matched 2D or 3D images may be used to determine relative locations of features of interest and to correlate the occurrence of features seen by light and electron microscopy. The matched 2D and 3D images may also be used to determine the structure of molecules of interest.

In some embodiments, subtomographic averages are computed for particles that are detected using the present technology. For example, the subtomographic averages may be used to calculate molecular envelopes. The subtomographic averages may also be used to calculate conformational states. The subtomographic averages may also be used to calculate 3D structures at near atomic resolution. The observed positions, orientations, and molecular conformations may be used to model the structure of the sample by segmentation. The observed positions, orientations, and molecular conformations may be used to model sample structure, including cellular structure, at the molecular, pseudo-atomic or atomic levels.

From the foregoing, it is appreciated that specific embodiments of the invention have been described herein for purposes of illustration, but that various modifications may be made without deviating from the scope of the invention. Accordingly, the invention is not limited except as by the appended claims.

### EXAMPLES

The following examples are intended to illustrate various embodiments of the present technology. As such, the specific embodiments discussed are not to be constructed as limitations on the scope of the present technology. It will be apparent to one skilled in the art that various equivalents, changes, and modifications may be made without departing from the scope of present technology, and it is understood that such equivalent embodiments are to be included herein. Further, all references cited herein are hereby incorporated by reference in their entirety, as if fully set forth herein.

### Example 1: Structural Properties of Calsequestrin

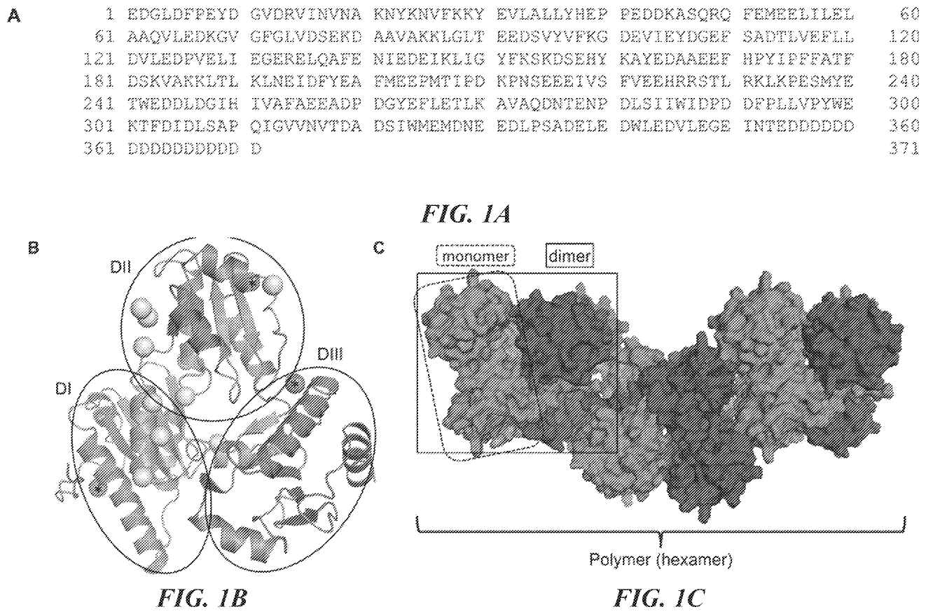

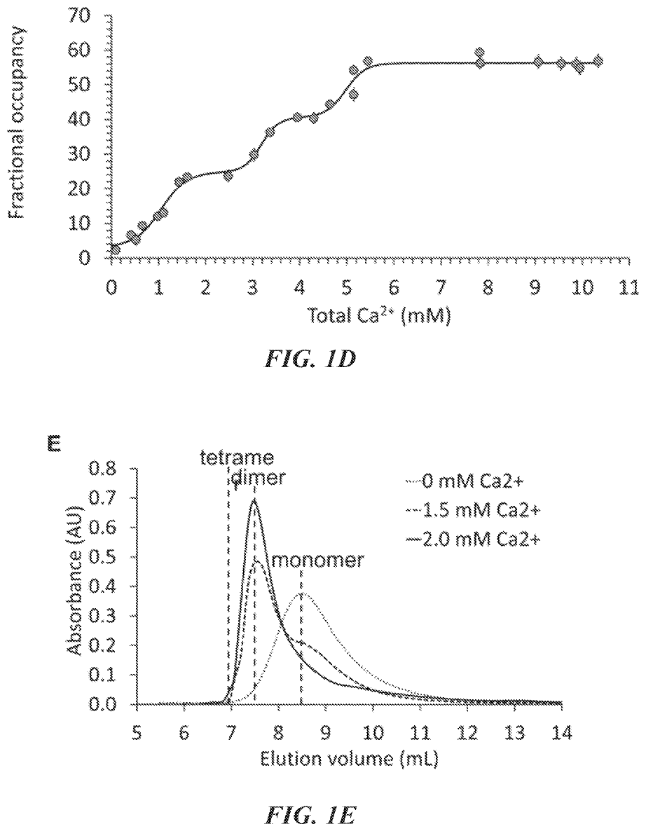

The microscopy probe may be based on M. musculus skeletal calsequestrin (MmCasq1). Structurally, calsequestrin consists of three mostly globular domains (Domain I, II, and III), along with an N-terminal arm and a polyanionic C-terminal tail (FIG. 1). Calsequestrin has a highly negatively-charged surface due to its disproportionately high D and E content (around 30% of amino acid residues) (FIG. 1A), which it uses to bind Ca²⁺ with a capacity of one-half its total D and E content (i.e., one-half its total negative charge), which in the case of mMCasq1 is 56 Ca²⁺ per MmCasq1 molecule (FIG. 1D). In contrast to its high capacity, MmCasq1 binds Ca²⁺ with a moderate affinity (Kd=1 mM) overall, as determined by the ratio of the off-rate (10-9 Ca²⁺ s−1) and on-rate (10-6 Ca²⁺ s⁻¹).

Calsequestrin's high-capacity Ca²⁺-binding capabilities, although based on charge neutralization, occur because of its unique Ca²⁺-dependent polymerization, which occurs in a stepwise manner. First, two monomers form a front-to-front dimer by way of N-terminal arm exchange and other protein-protein interactions, both Ca²⁺-dependent and independent (FIG. 1C). The resulting N-terminal interface between the dimeric partners allows cooperative Ca²⁺ coordination in a symmetric fashion. The front-to-front dimer can interact with another front-to-front dimer through their respective C-terminal ends to form a tetramer through a Ca²⁺-driven mechanism, and eventually, a polymer (FIG. 1C). Calsequestrin remains capable of binding calcium in both its monomeric and oligomeric state, seen in calsequestrin's multi-phasic Ca²⁺-binding profile (FIG. 1D). Calsequestrin may form one-dimensional polymers (FIG. 1C), with some off-branching tendrils.

A microscopy probe comprising concatenated calsequestrin monomers (i.e. dimers, trimers, tetramers in a single polypeptide sequence) will be developed as part of this technology.

### Example 2: Structural Mechanism for Lanthanide Luminescence

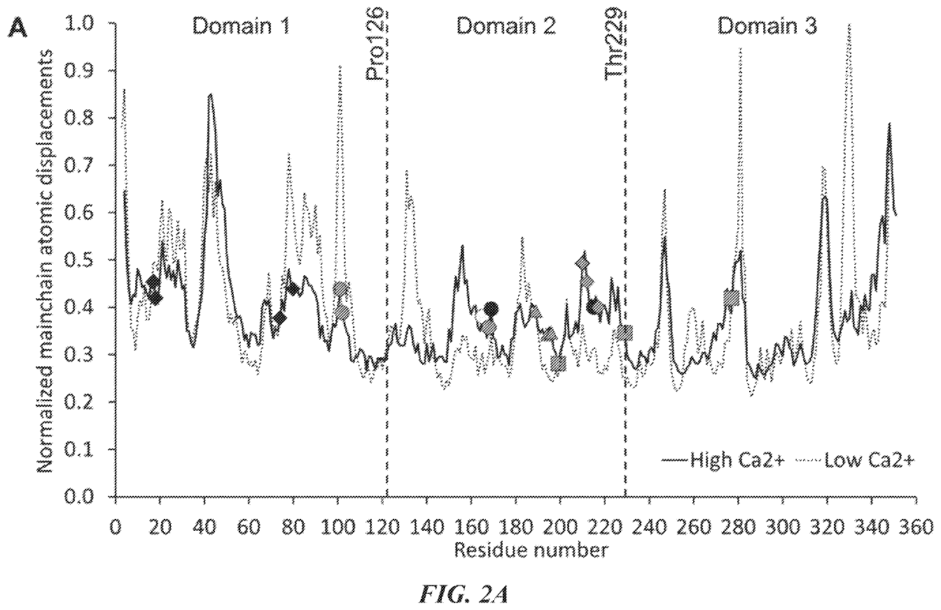

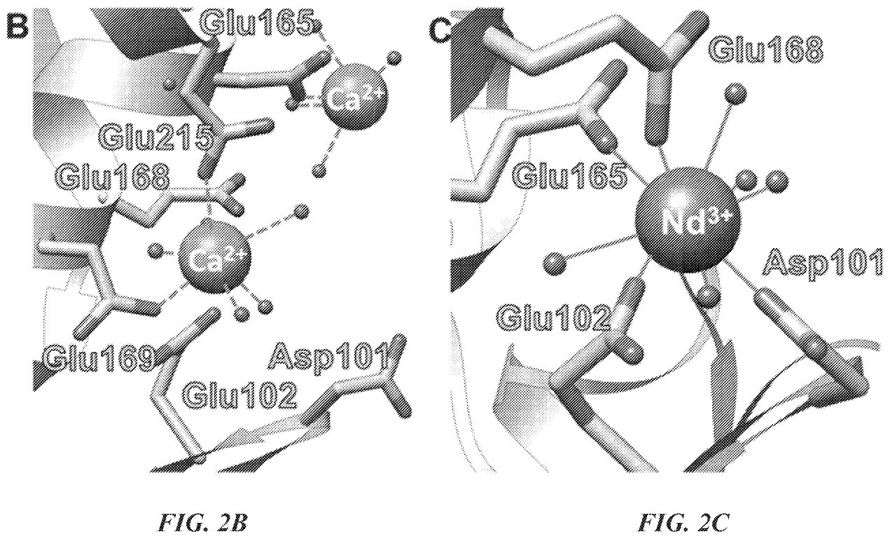

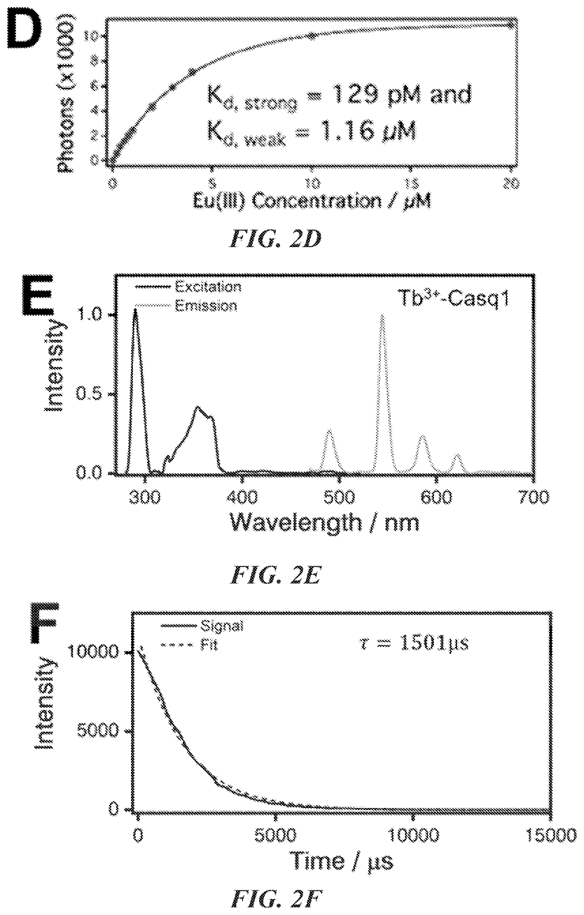

Calsequestrin contains what have been identified as three to four structural Ca²⁺-binding sites, named high-affinity site A (FIG. 2A, four black diamonds), high-affinity site B (FIG. 2A, three dark gray squares), high-affinity site C (FIG. 2A, three dark gray diamonds), and high-affinity site D (FIG. 2A, three dark gray triangles). As shown by the plot of normalized mainchain atomic displacements obtained from X-ray crystallographic data (FIG. 2A) of the low Ca²⁺ (structural Ca²⁺ only; dotted line) and high Ca²⁺ (~17 Ca²⁺ per Casq1 molecule; solid line) forms of Casq1, Casq1 has multiple disordered regions that undergo a disordered-to-ordered transition upon binding Ca²⁺, Sr²⁺, and Ba²⁺.

Lanthanides, which have traditionally been used in X-ray crystallography to probe Ca²⁺ binding sites due to their similar ionic radii and coordination chemistry to Ca²⁺ (e.g., 112 pm radius for 8-coordinate Nd³⁺ vs. 111 pm radius for 8-coordinate Ca²⁺) but higher scattering power due to their larger number of electrons (e.g., 5π electrons in Nd³⁺ vs. 18 in Ca²⁺), also bind to Casq1. The crystal structure of Casq1 that has at least two sites unambiguously occupied by Pr³⁺ and Nd³⁺ has been solved. Due to the higher charge density than Ca²⁺, both Pr³⁺ and Nd³⁺ change a binuclear Ca²⁺ binding site (dark gray squares in FIG. 2A are inner sphere ligands for Ca²⁺, but outer sphere for Nd³⁺, whereas the light gray circle is inner sphere for both Ca²⁺ and Nd³⁺ and the dark gray circles are outer sphere for Ca²⁺ but inner sphere for Nd³⁺; Ca²⁺ are large spheres in FIG. 2B) into a mononuclear lanthanide binding site (Nd³⁺ shown as large sphere in FIG. 2C). The higher positive charge density of lanthanides leads to tighter binding by Casq1 over Ca²⁺, as shown by picomolar affinities for Eu³⁺ compared to millimolar affinities for Ca²⁺, which is reflected in the crystal structure where Nd³⁺ is more highly chelated by four Casq1 sidechains than either of the two Ca²⁺ in the native bimetallic site, which are bound in an inner sphere coordination mode by one sidechain for one Ca²⁺ and two sidechains for the second Ca²⁺.

Binding of lanthanides by Casq1 breaks the symmetry of the lanthanide's ligand field, thereby enhancing the intensity of the lanthanide's characteristic symmetry-forbidden f-f transitions (FIG. 2D). Due to the phosphorescent nature of lanthanide luminescence, their luminescence is long lived (FIG. 2E), which allows for time-gated experiments. The tight binding of lanthanides by Casq1 in conjunction with lanthanide luminescence is what allows it to be a useful luminescent probe since the tight binding ensures that bound lanthanides will stay bound at any practical Casq1 concentration in the absence of excess lanthanide in solution.

### Example 3: HsCasq1 and MmCasq1 as Luminescent Microscopy Probes

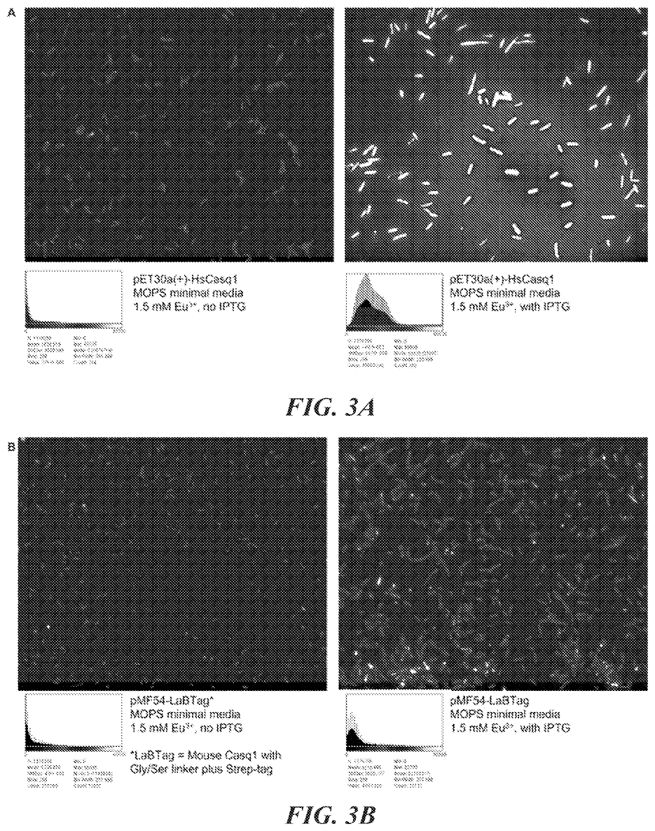

Because human Casq1 (HsCasq1) and MmCasq1 are genetic constructs, they were expressed inside living cells grown in the presence of sub-millimolar quantities of lanthanides like Eu³⁺ in an innovative defined cell culture medium that has disodium α-glycerophosphate instead of sodium phosphate in order to prevent precipitation of lanthanides as lanthanide phosphates. As shown in FIG. 3, both E. coli expressing HsCasq1 (FIG. 3A) and P. aeruginosa PAO1 (FIG. 3B) expressing a MmCasq1 with a N-terminal Gly/Ser linker and Strep tag showed markedly increased Eu³⁺ luminescence over non-specific signal of non-expressing cells. The genetic expressibility of HsCasq1 and MmCasq1 allows it to be fused to proteins of interest, and because it can be fused, it can allow luminescence imaging inside a cell without having to resort to disruptive means of introducing luminescent nanoparticles into a cell or relying on non-specific chemical means of labeling proteins inside a cell post-expression.

Next, the luminescence of MmCasq1 and HsCasq1 was imaged under cryogenic conditions before imaging on the electron microscope. Luminescent imaging of MmCasq1 and HsCasq1 is possible under cryogenic conditions (considering X-ray diffraction under cryogenic conditions shows a correctly folded protein with bound lanthanides), and it often even works better under cryogenic conditions than room temperatures because of the loss of non-radiative decay at cryogenic temperatures, which lengthens the luminescence lifetime of MmCasq1 and HsCasq1.

In cases where the sample is too thick for transmission electron microscopy, the cryo-luminescence of a luminescent metal binding protein or peptide enabled light-guided focused ion beam (FIB) milling of the sample. Because MmCasq1 and HsCasq1 exhibit lengthened luminescence lifetimes under cryogenic conditions, it will be a superior probe for time-resolved imaging of cells to guide FIB milling.

### Example 4: Cryo-ET Using MmCasq1

The electron dense nature of lanthanides and actinides bound by MmCasq1 allowed the MmCasq1 to work not only as a luminescent probe, but also as a genetically encoded electron dense contrasting agent in electron microscopy. Luminescent metal binding proteins and peptides like MmCasq1 enable Correlative Light and Electron Microscopy (CLEM), a two-part technique that utilizes both luminescent (light) microscopy and electron microscopy (EM) and correlates the results from these complementary imaging modalities. The dual nature of the genetically encoded MmCasq1, which is both luminescent and electron dense, allows a single fusion protein tag to identify the target protein under both imaging modalities.

CLEM can be performed with either an SEM or a TEM. In the case of SEM, the specimen may be imaged by serial block face milling. It can also be imaged with “integrated CLEM”, in which the light microscope is integrated into the SEM so that the light microscopy and block face imaging data are collected in tandem.

In the case of CLEM with TEM, cryogenic light microscopy (in this case, lanthanide luminescence) is first used to find the region of interest of the specimen. The light microscope can also be used to select a region of interest within the volume of a frozen cell for TEM. The sample is then transferred into the TEM, where micrographs or a tilt series are collected on the region of interest. The “tilt-series” is a series of micrographs that are collected as set positions as the stage is rotated+/−60-70 degrees in discrete steps (for example 2 or 3 degrees). This tilt series is then used to construct a 3D tomogram, i.e., a 3D image of the sample. The 2D or 3D light microscopy data are then overlaid or superposed on the 2D micrograph or 3D tomographic volume, so that these complementary set of data can be correlated and analyzed. Specifically, the electron dense fusion tag can then be visualized within the 3D volume to determine the exact location of the Mm Casq1, and thus the protein to which it is fused. In some cases, the overlay of the luminescent signal can be used to aid the search.

Frequently, the sample may be too thick to image by TEM. In these cases, the sample may be thinned. If thinning is needed, the luminescent signal can be used to guide FIB-milling in the SEM or “integrated SEM”, to produce thinned specimens, including cross sections through the cell (lamellae), or lift outs. Following FIB milling, the luminescent signal can be used yet again to guide subsequent data collection in the transmission electron microscope, as described above.

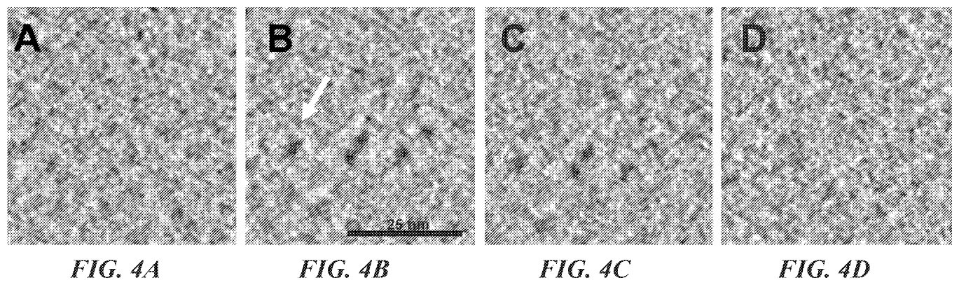

Purified MmCasq1 (0.1 mg/ml) was applied to Quantifoil R2/1 grids, then blotted and plunge frozen in liquid propane (Vitrobot Mark IV). Tilt series were taken using the Montana State University Talos Arctica operated at 200 kV with a Gatan K3 camera. Four successive sections along Z (each 4.6 nm thick) through a representative sub tomographic volume (50×50×20 nm) are presented in FIGS. 4A-4D. A cluster of five MmCasq1 probes is clearly visible as black punctate points 2.3 nm in diameter, extending left to right across FIGS. 4B and 4C. The position of the left most particle is denoted by the arrow in FIG. 4B. Acquisition parameters were as follows: magnification 36,000×; dose symmetric tilt range+/−60 degrees; tilt increment 3 degrees; total dose 60 e-per square Angstrom; pixel size 0.115 nm/px; defocus −3.0 μm. Tomograms were reconstructed using IMOD 4.11.

Eu-loaded MmCasq1 was easily visible as a 2-3 nm electron dense feature with the symmetry of a MmCasq1 dimer when looking at purified protein (FIG. 4A-4D). This, combined with luminescent cellular imaging of overexpressed Eu-MmCasq1, shows that MmCasq1's luminescent properties can be used not only to guide FIB milling, but also to enable target protein localization in the subsequent tomograms with great precision. MmCasq1 may be detectable in complex protein mixtures (whole cells, cellular cross sections, and cell lysates).

### Example 5: CLEM of MmCasq1 and HsCasq1

Cells were transformed or gene edited with a HsCasq1 or MmCasq1 fusion, and expressed under desired conditions, such as the example shown in FIG. 3. Next, cells will be labeled with lanthanides of interest and frozen on electron microscope grids. Luminescence will then be observed by cryo-light microscopy and used to guide FIB milling of cross sections (lamelli) through the cells, or lift outs. Then phosphorescence will be observed in the milled cross sections or lift outs to select regions of interest for acquisition of TEM micrographs or tilt-series (tomograms). This will be followed by imaging in the electron microscope, which will provide additional, complementary information. Micrographs and tomograms will be searched for electron dense particles to localize individual fusion protein molecules.

In some instances, the search will be limited to subvolumes initially identified by light microscopy or super-resolution light microscopy at the single molecule level. 2D or 3D templates may be used to search the EM subvolumes. Once MmCasq1 or HsCasq1 fusions are localized within the subvolumes, the immediate vicinity may be searched for the molecule of interest, again using template matching algorithms or other strategies, to yield information on the orientation, assembly state and conformation of the target protein within the micrograph or tomogram. Subtomographic averages may be computed for particles or particles classes to determine molecular envelopes, conformational states and/or 3D structures at near atomic resolution. In some embodiments, the positions, orientations, and molecular conformations may be thus observed, allowing cellular structure to be modeled at the (pseudo-)atomic level. The light and electron microscopy data may then be overlayed, allowing the data to be analyzed together.

### Additional Examples

Several aspects of the present technology are set forth in the following examples:
1. A microscopy probe comprising a luminescent metal binding protein, wherein:
   the microscopy probe is configured to be detected using a light microscope and an electron microscope; and
   the luminescent metal binding protein is configured to chelate a luminescent metal.
2. The microscopy probe of example 1, wherein the luminescent metal has an atomic number of at least 57.
3. The microscopy probe of any of examples 1-2, wherein the luminescent metal binding protein comprises more than 20% N and Q amino acids.
4. The microscopy probe of any of examples 1-3, wherein the luminescent metal binding protein comprises an amino acid sequence that is at least 80% identical to SEQ ID NO: 1 or 2.
5. The microscopy probe of any of examples 1-4, wherein the luminescent metal binding protein is configured to bind to more than 10 luminescent metals.
6. The microscopy probe of any of examples 1-5, wherein the luminescent metal is a lanthanide or actinide.
7. The microscopy probe of any of examples 1-6, wherein the luminescent metal is selected from a group consisting of Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, U³⁺, U⁴⁺, U⁵⁺, and U⁶⁺.
8. The microscopy probe of any of examples 1-7, wherein the microscopy probe is configured to be detected using correlated light and electron microscopy.
9. A polynucleotide encoding the microscopy probe of any of examples 1-8.
10. A vector comprising the polynucleotide of example 9.
11. A method of correlated light and electron microscopy (CLEM) comprising detecting a microscopy probe using a light microscope and an electron microscope, wherein the microscopy probe comprises a luminescent metal binding protein that is configured to chelate a luminescent metal.
12. The method of example 11, wherein the luminescent metal has an atomic number greater than 57.
13. The method of any of examples 11-12, wherein the luminescent metal is a lanthanide.
14. The method of any of examples 11-13, wherein the luminescent metal is selected from a group consisting of Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, U³⁺, U⁴⁺, U⁵⁺, and U⁶⁺.
15. The method of any of examples 11-15, wherein the luminescent metal binding protein comprises more than 20% N and Q amino acids.
16. The method of any of examples 11-15, wherein the luminescent metal binding protein is a ortholog, paralog, or analog of calsequestrin.
17. The method of any of examples 11-16, wherein the luminescent metal binding protein is a mouse or human calsequestrin.
18. The method of any of examples 11-17, wherein the luminescent metal binding protein has a sequence 80% identical to SEQ ID NO: 1 or 2.
19. The method of example 11-18, wherein the microscopy probe is genetically encoded and expressed in a cell or multicellular organism.
20. The method of example 19, wherein the light microscope is used to detect the microscopy probe in the cell or multicellular organism and the electron microscope is used to detect the microscopy probe in the cell or multicellular organism.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the microscopy probes described herein may be combined with any of the features of the other microscopy probes described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with luminescent metal binding proteins and methods of using the luminescent metal binding proteins have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A microscopy probe comprising a luminescent metal binding protein, wherein:
the microscopy probe is configured to be detected using a light microscope and an electron microscope; and
the luminescent metal binding protein is configured to chelate a luminescent metal.

2. The microscopy probe of claim 1, wherein the luminescent metal has an atomic number of at least 57.

3. The microscopy probe of any of claims 1-2, wherein the luminescent metal binding protein comprises more than 20% N and Q amino acids.

4. The microscopy probe of any of claims 1-3, wherein the luminescent metal binding protein comprises an amino acid sequence that is at least 80% identical to SEQ ID NO: 1 or 2.

5. The microscopy probe of any of claims 1-4, wherein the luminescent metal binding protein is configured to bind to more than 10 luminescent metals.

6. The microscopy probe of any of claims 1-5, wherein the luminescent metal is a lanthanide or actinide.

7. The microscopy probe of any of claims 1-6, wherein the luminescent metal is selected from a group consisting of Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, U³⁺, U⁴⁺, U⁵⁺, and U⁶⁺.

8. The microscopy probe of any of claims 1-7, wherein the microscopy probe is configured to be detected using correlated light and electron microscopy.

9. A polynucleotide encoding the microscopy probe of any of claims 1-8.

10. A vector comprising the polynucleotide of claim 9.

11. A method of correlated light and electron microscopy (CLEM) comprising detecting a microscopy probe using a light microscope and an electron microscope, wherein the microscopy probe comprises a luminescent metal binding protein that is configured to chelate a luminescent metal.

12. The method of claim 11, wherein the luminescent metal has an atomic number greater than 57.

13. The method of any of claims 11-12, wherein the luminescent metal is a lanthanide.

14. The method of any of claims 11-13, wherein the luminescent metal is selected from a group consisting of Ce³⁺, Pr³⁺, Nd³⁺, Pm³⁺, Sm³⁺, Eu³⁺, Gd³⁺, Tb³⁺, Dy³⁺, Ho³⁺+, Er³⁺, Tm³⁺, Yb³⁺, U³⁺, U⁴⁺, U⁵⁺, and U⁶⁺.

15. The method of any of claims 11-15, wherein the luminescent metal binding protein comprises more than 20% N and Q amino acids.

16. The method of any of claims 11-15, wherein the luminescent metal binding protein is a ortholog, paralog, or analog of calsequestrin.

17. The method of any of claims 11-16, wherein the luminescent metal binding protein is a mouse or human calsequestrin.

18. The method of any of claims 11-17, wherein the luminescent metal binding protein has a sequence 80% identical to SEQ ID NO: 1 or 2.

19. The method of claim 11-18, wherein the microscopy probe is genetically encoded and expressed in a cell or multicellular organism.

20. The method of claim 19, wherein the light microscope is used to detect the microscopy probe in the cell or multicellular organism and the electron microscope is used to detect the microscopy probe in the cell or multicellular organism.
